(19) United States
(12) Patent Application Publication
(10) Pub. No.: US 2026/0102322 A1
(43) Pub. Date: Apr. 16, 2026

(54) ORAL CARE COMPOSITIONS COMPRISING CARBOXYLIC ACIDS

(71) Applicant: The Procter & Gamble Company, Cincinnati, OH (US)

(72) Inventors: Samuel James ST. JOHN, Cincinnati, OH (US); Andrew Frederic Groth, Mason, OH (US)

(21) Appl. No.: 19/352,769

(22) Filed: Oct. 8, 2025

Related U.S. Application Data
(60) Provisional application No. 63/705,731, filed on Oct. 10, 2024.

(51) Int. Cl. A61K 8/21; A61K 8/19; A61K 8/24; A61K 8/25; A61K 8/27; A61K 8/34; A61K 8/365; A61K 8/44; A61K 8/49; A61K 8/73; A61Q 11/00

(52) U.S. Cl. CPC A61K 8/21; A61K 8/19; A61K 8/24; A61K 8/25; A61K 8/27; A61K 8/345; A61K 8/365; A61K 8/44; A61K 8/49; A61K 8/73; A61Q 11/00; A61K 2800/28; A61K 2800/30

(57) ABSTRACT

An oral care composition includes a stannous ion source, a source of ethylenediaminetetraacetate, and a ligand comprising oxalate, gluconate, citrate, a salt thereof, or a combination thereof. The oral care composition may provide efficacious fluoride uptake and stain removal.

### FIELD

The present disclosure is directed to oral care compositions comprising oxalic, malonic acid, ethylenediaminetetraacetic acid their salts with tin and fluoride, or combinations thereof that have an improved delivery of stannous and fluoride to tooth surfaces. The present disclosure is also directed to methods for treating and/or preventing demineralization and enhancing remineralization comprising directing a user to apply an oral care composition comprising carboxylic acids to an oral cavity.

### BACKGROUND

Oral care compositions have included antimicrobial agents, such as tin ions, to counter oral bacteria and to prevent and treat conditions caused by bacteria in the oral cavity, such as formation of dental plaque and calculus. The formation of dental plaque and calculus and failure to stop their proliferation are the primary cause of dental caries, gingivitis, periodontal disease, and tooth loss. Additionally, tin ions can deposit on surfaces in the oral cavity to provide protective functions, such as antierosion or antisensitivity benefits.

However, tin can be challenging to properly formulate in oral care compositions due to reactivity between tin and other components of oral care compositions. Under-stabilizing or over-stabilizing tin can lead to lower availability of tin ions to provide the desired benefit. For example, if the tin is under-stabilized, the tin can react with other components of the oral care composition, such as silica, water, etc., which can lead to a lower amount of available tin ions. In contrast, if the tin is over-stabilized or the chelant-tin chelation is too strong, the tin ions will be tied up while in the oral cavity, which can also lead to a lower amount of bioavailable tin ions to produce the desired oral care benefit.

Thus, the tin-chelant ratio and binding affinity must be carefully balanced to maximize the amount of available tin ions. As such, there is a need for oral care compositions comprising a high amount of available tin ions that are highly reactive/bioavailable for the desired product benefit.

Additionally and unfortunately, many cationic antimicrobial agents can also stain the dental hard tissues when they are used to treat gingivitis. As such, there is a need for anti-gingivitis agents that do not pose some of the stability concerns of metal ion sources or do not produce dental stain. Accordingly, there is a need for an oral care composition that can be used to effectively deliver stannous and fluoride to oral hard tissues while controlling dental stain.

### SUMMARY

Disclosed is an oral care composition that includes a stannous ion source, a source of ethylenediaminetetraacetate, and a ligand comprising a dicarboxylate source. The dicarboxylate source can be selected from oxalates, gluconates, citrates, and combinations thereof. The oral care composition may have a molar ratio of stannous ions to EDTA in a range of 1:0.5 to 1:2.5 and/or a pH of 6 to 8. The oral care composition provides efficacious fluoride uptake and tooth stain removal. The oral care composition may be used to enhance fluoride uptake, protecting a hydroxyapatite surface from citric acid attack, and/or removing tooth stain.

### BRIEF DESCRIPTION OF THE FIGURES

FIG. 1 is a graph showing the fluoride uptake and HAP dissolution results for various compositions.

### DETAILED DESCRIPTION

Embodiments of the present invention are directed to oral care whitening compositions that have dicarboxylic acid, such as oxalic acid, malonic acid, ethylenediaminetetraacetic acid, or combinations thereof and provide an unexpectedly high reduction of hydroxyapatite solubility in citric acid, while simultaneously delivering an unexpectedly high amount of fluoride to demineralized human enamel and removing a large amount of dental stain.

Although the initial process related to both caries and dental erosion begins with teeth being subjected to acid attack, the subsequent stages of each process are quite distinct. Dental erosion is a process that generally initiates on facial surfaces of teeth on which plaque is not present, while caries occurs under plaque-coated surfaces, where relatively constant, low-level acid challenges penetrate through the surface of the teeth and create subsurface lesions while allowing the surface to remain intact. In the case of dental erosion, excessive exposure to dietary acids causes the surfaces of the teeth then begin to soften, resulting in tooth loss. Although fluoride is recognized to strengthen acid damaged enamel during remineralization, fluoride provides poor protection against dietary acids.

SnF₂ is a well-established anticaries agent that is unique among the fluoride sources used in over-the-counter dentifrices because of the stannous ion. Stannous deposits onto the tooth surface at both calcium and phosphate sites forming a thin layer of insoluble mineral precipitates. These precipitates slow the attack of dietary acids by stabilizing the enamel surface thus helping to prevent the loss of enamel to erosive processes. The ability of stannous to reach and then react with the enamel surface depends upon the choice of stabilizers in the oral care composition.

The unique properties of small molecule mono-, di-, tri-, and tetra-carboxylic acids, like gluconic acid, oxalic acid, citric acid, and ethylenediaminetetraacetic acid, allow them to be highly effective stabilizing ligands in a particular pH range and in a particular molar ratio to stannous. A specific combination of these small-molecule stabilizers allows for adequate shelf stability and high bioavailability. While not wishing to be bound by theory, it is believed that the disclosed oral care compositions of the present invention provide an unexpectedly high erosion benefit in comparison to less well stabilized compositions defined by an optimum ratio of Sn to mono-to poly-carboxylic acid stabilizers.

Additionally, while the use of cationic antimicrobial agents can provide many benefits when applied to the oral cavity, as described herein, cationic antimicrobial agents can also contribute to surface staining on teeth. Oral hard surface stains can be caused by interaction between (1) cation-crosslinked proteins and/or extracellular polysaccharides and (2) colored porphyrins and organic and/or inorganic chromophores, such as metal ions, which can form a colored matrix. Disrupting this colored matrix can facilitate stain removal.

Chemical whitening agents loosen the interactions of the compounds in this colored matrix to dislodge it from the oral hard tissue surfaces. While not wishing to be bound by theory, it is believed that chemical whitening agents, such as dicarboxylic acid or ethylenediaminetetraacetic acid, can act as solubilizing ligands for the porphyrins and/or chromophores to remove stain from the surface. Furthermore, manipulating the pH and ionic strength of the disclosed oral care composition can further reduce the strength of the electrostatic bonds by protonating anionically charged moieties or by reducing the potential of the electrostatic double layer further facilitating the solubilization of cationic moieties by solubilizing ligands.

In total, the unique properties of polycarboxylic acid compounds, such as oxalic acid or ethylenediaminetetraacetic acid, allow them to be highly effective stabilizing ligands for porphyrins and/or chromophores in a particular pH range. As such, the present invention is directed to oral care compositions that provide an unexpectedly high whitening benefit in a pH range in which convention whitening agents cannot be used.

### Definitions

“Oral care composition” means a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, emulsion, mouth rinse, mousse, foam, mouth spray, lozenge, chewable tablet, chewing gum, tooth whitening strips, floss and floss coatings, breath freshening dissolvable strips, unit-dose composition, fibrous composition, or denture care or adhesive product. The oral care composition may also be incorporated onto strips or films for direct application or attachment to oral surfaces, such as tooth whitening strips. Examples of emulsion compositions include the emulsions compositions of U.S. U.S. Pat. No. 11,147,753, jammed emulsions, such as the jammed oil-in-water emulsions of U.S. Pat. No. 11,096,874. Examples of unit-dose compositions include the unit-dose compositions of U.S. Patent Application Publication No. 2019/0343732.

“Dentifrice composition” means tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy.

The carriers or excipients useful in embodiments of the present invention can include the usual and conventional components of mouthwashes or mouth rinses. Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health, e.g., toothpaste, mouth rinse, floss, toothpicks, spray, water irrigator, massager.

The term “total water content” as used herein means both free water and water that is bound by other ingredients in the oral care composition.

For the purpose of this description, the relevant molecular weight (MW) to be used is that of the material added when preparing the composition, e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e., room temperature) unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, and so forth.

Several types of ranges are disclosed in relation to embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care composition can include a variety of active and inactive ingredients, such as, for example, but not limited to a hops extract, a dicarboxylic acid, a tin ion source, a calcium ion source, water, a fluoride ion source, zinc ion source, one or more polyphosphates, humectants, surfactants, other ingredients, and the like, as well as any combination thereof, as described below. The section headers below are provided for organization and convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

Humulus lupulus

Oral care compositions of the present invention can comprise hops. The hops can comprise at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into trans-isohumulone can be represented by Formula III.

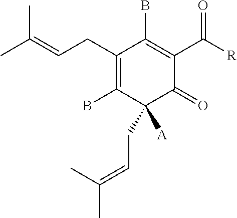

Formula I. Hops Alpha Acids. A is the acidic hydroxyl functional group in the alpha position. B arc the acidic hydroxyl functional groups in the beta position, and R is an alkyl functional group.

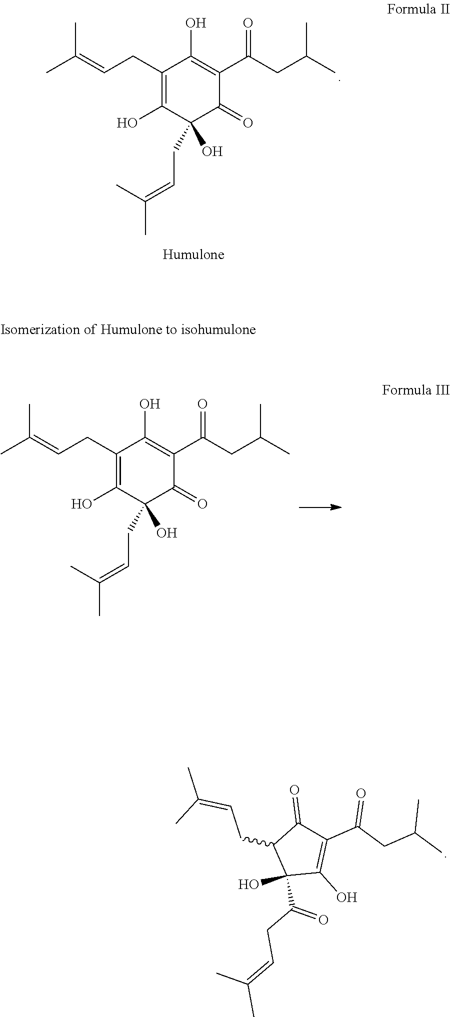

Suitable hops beta acids can include lupulone, adlupulone, colupulone, and/or mixtures thereof. A suitable hops beta acid can include a compound a described in Formula IV, V, VI, and/or VII.

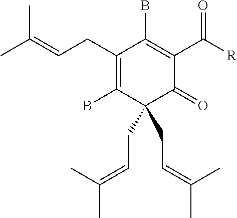

Formula IV. Hops Beta Acids. B are the acidic hydroxyl functional groups in the beta position and R is an alkyl functional group.

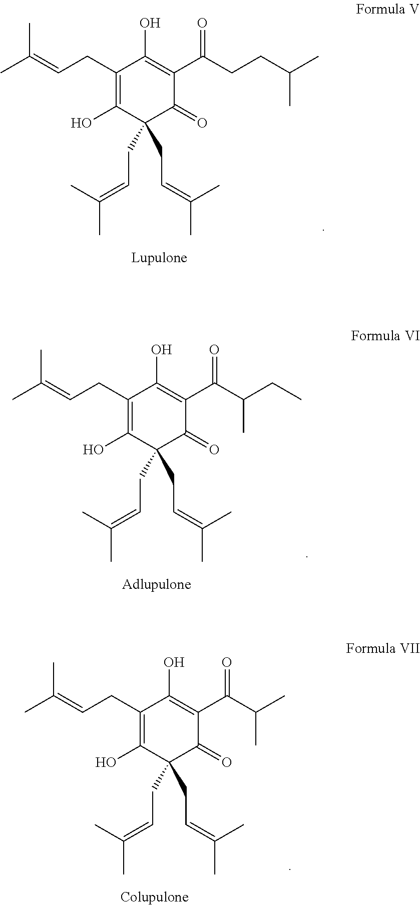

While hops alpha acids can demonstrate some antibacterial activity, hops alpha acids also have a bitter taste. The bitterness provided by hops alpha acids can be suitable for beer, but they are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent.

A natural hops source can comprise from about 2% to about 12%, by weight of the hops source, of hops beta acids depending on the variety of hops. Hops extracts used in other contexts, such as in the brewing of beer, can comprise from about 15% to about 35%, by weight of the extract, of hops beta acids. The hops extract desired herein can comprise at least about 35%, at least about 40%, at least about 45%, from about 35% to about 95%, from about 40% to about 90%, or from about 45% to about 99%, of hops beta acids. The hops beta acids can be in an acidic form (i.e. with attached hydrogen atom(s) to the hydroxyl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract from another plant, such as a species from genus Magnolia. The hops compounds can be combined with or free from triclosan.

The hops, such as the hops beta acid, can be provided by a suitable hops extract, the hops plant itself, or a synthetically derived compound. The hops, such as hops beta acid, can be provided as neutral, acidic compounds, and/or as salts with a suitable counter ion, such as sodium, potassium, ammonia, or any other suitable counter ion.

The hops can be provided by a hops extract, such as an extract from Humulus lupulus with at least 35%, by weight of the extract, of hops beta acid and less than 1%, by weight of the hops extract, of hops alpha acid.

### Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula VIII-A, Formula VIII-B, and/or Formula VIII-C.

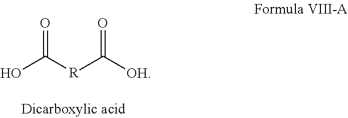

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

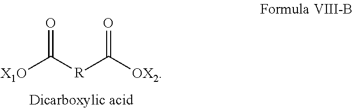

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

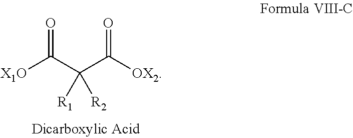

R₁ can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula VIII-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula VIII-D.

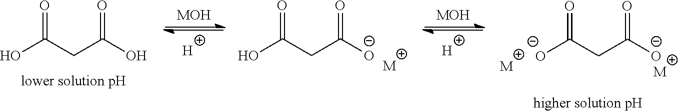

Formula VIII-D. Acid-Base Properties of Dicarboxylic Acid, wherein M is any metal.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid:

monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

Suitable dicarboxylic acid compounds include malonic acid, methylmalonic acid, tartronic acid, malic acid, dimethylmalonic acid, mesoxalic acid, dihydroxymalonic acid, oxalic acid, salts thereof, or combinations thereof. These dicarboxylic acid compounds are particularly suitable as these compounds have been shown to have an unexpectedly high whitening benefit. While not wishing to be bound by theory, it is believed that particular dicarboxylic acid compounds have an unexpectedly high affinity to certain cationic crosslinking agents typically found in the colored matrix on the oral hard tissue surfaces, thereby resulting in the removal of stain from the surface.

Suitable dicarboxylic acid compounds include dicarboxylic acids described by Formula VIII-A, wherein R is null, comprises a methylene or ethylene with one or two substitutions, and/or an acetyl group.

Without being bound by theory, it is hypothesized that the whitening efficacy of the dicarboxylics acids and their corresponding anions is driven by the ability of the dicarboxylic acid to reach and remove cationic bridges between chromophores and the tooth surface as well as chromophores and the pellicle proteins.

The oral care composition can comprise from about 0.0001% to about 25%, from about 0.01% to about 20%, from about 0.1% to about 15%, from about 0.1% to about 10%, or from about 1% to about 5%, by weight of the oral care composition, of dicarboxylic acid.

### Ethylenediaminetetraacetic Acid (EDTA)

The oral care composition comprises ethylenediaminetetraacetic acid, which can be provided by its acid form or by its monobasic, dibasic, tribasic, or tetrabasic forms and adjusted to the appropriate composition pH using pH adjusters. In some embodiments, combinations of any of the forms can be used to create the appropriate buffer system at the appropriate pH of the composition. The basic forms can use any number of appropriate metal salts such as sodium or potassium.

The oral care composition can comprise from about 0.5% to about 5%, from about 1% to about 3%, from about 1% to about 2.5%, from about 0.5% to about 2.5%, from about 0.5% to about 2.0%, or from about 1% to 2% EDTA anion. In another embodiment, the oral care composition may be substantially free of, essentially free of, or free of EDTA.

Diethylenetriaminepentaacetic acid (DTPA)

The oral care composition comprises diethylenetriaminepentaacetic acid, which can be provided by its acid form or by its monobasic, dibasic, tribasic, tetrabasic, or pentabasic forms and adjusted to the appropriate composition pH using pH adjusters. DTPA is illustrated below:

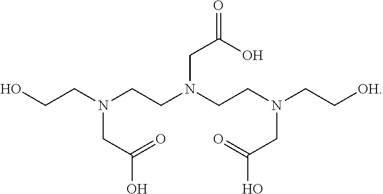 [SMILES: O=C(O)CN(CCO)CCN(CCN(CCO)CC(=O)O)CC(=O)O]

Alternatively, combinations of any of the forms can be used to create the appropriate buffer system at the appropriate pH of the composition. The basic forms can use any number of appropriate metal salts such as sodium or potassium.

The oral care composition can comprise from about 0.5% to about 5%, from about 1% to about 3%, from about 1% to about 2.5%, from about 0.5% to about 2.5%, from about 0.5% to about 2.0%, or from about 1% to 2% DTPA anion. In another embodiment, the oral care composition may be substantially free of, essentially free of, or free of DTPA.

### Etidronic Acid (HEDP)

The oral care composition comprises etidronic acid, which can be provided by its acid form or by its monobasic, dibasic, tribasic, or tetrabasic forms and adjusted to the appropriate composition pH using pH adjusters. HEDP is illustrated below:

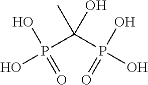

Alternatively, combinations of any of the forms can be used to create the appropriate buffer system at the appropriate pH of the composition. The basic forms can use any number of appropriate metal salts such as sodium or potassium.

The oral care composition can comprise from about 0.5% to about 5%, from about 1% to about 3%, from about 1% to about 2.5%, from about 0.5% to about 2.5%, from about 0.5% to about 2.0%, or from about 1% to 2% HEDP anion. In another embodiment, the oral care composition may be substantially free of, essentially free of, or free of HEDP.

### Fluoride

The oral care composition can comprise fluoride, which can be provided by a fluoride ion source. The fluoride ion source can comprise one or more fluoride containing compounds, such as stannous fluoride, sodium fluoride, titanium fluoride, calcium fluoride, calcium phosphate silicate fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or be free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, K, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15%, by weight of the oral care composition, of metal and/or a metal ion source.

In various embodiments, the oral care composition may include residual iron in a range of from about 1 to about 1,000 ppm, from about 10 ppm to about 500 ppm, or from about 10 ppm to about 200 ppm. Residual iron refers to iron that is present in the composition but is not deliberately added to the composition. It is meant to include compositions whereby the iron is present only as an impurity of one of the other materials deliberately added (e.g., silica) or that entered the composition during the manufacturing process.

### Tin

An oral care composition according to embodiments of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Antibacterial Agents

The oral care composition can comprise one or more antibacterial agents. Suitable antibacterial agents include any molecule that provides antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition can comprise from about 0.01% to about 10%, from about 1% to about 5%, or from about 0.5% to about 15% of an antibacterial agent.

### Bioactive Materials

The oral care composition can also include bioactive materials suitable for the remineralization of a tooth. Suitable bioactive materials include bioactive glasses, Novamin™, Recaldent™, hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise from about 0.01% to about 20%, from about 0.1% to about 10%, or from about 1% to about 10% of a bioactive material by weight of the oral care composition.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, from about 0.5% to about 1.5%, or from about 0.3% to about 0.6%, by weight of the oral care composition. Alternatively, the oral care composition can be essentially free of, substantially free of, or free of zinc.

### Potassium

The oral care composition can comprise potassium, which can be provided by a potassium ion source. The potassium ion source can comprise one or more potassium containing compounds, such as potassium nitrate, potassium fluoride, potassium chloride, or combinations thereof.

The oral care composition can comprise from about 0.01% to about 10%, from about 0.2% to about 1%, from about 0.4% to about 1%, or from about 0.3% to about 0.6%, by weight of the oral care composition, of potassium and/or potassium ion source. Alternatively, the oral care composition can be essentially free of, substantially free of, or free of potassium.

### Quaternary Ammonium Compound

The oral care composition can include quaternary ammonium compound. The quaternary ammonium compounds in the compositions of embodiments of the present invention can include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Cetylpyridinium chloride, cetyl pyridinium fluoride, tetradecylpyridinium chloride, N-tetradecyl-4-ethyl pyridinium chloride, domiphen bromide, benzalkonium chloride, benzethonium chloride, methyl benzethonium chloride, dodecyl trimethyl ammonium bromide, dodecyl dimethyl (2-phenoxyethyl) ammonium bromide, benzyl dimethoxystearyl ammonium chloride, quaternized 5-amino-1,3-bis(2-ethyl-hexyl)-5-methyl hexa hydropyrimidine, lauryl trimethylammonium chloride, cocoalkyl trimethylammonium chloride, cetyl trimethylammonium bromide, di-isobutylphenoxyethyl-dimethylbenzylammonium chloride, dodecyl trimethyl ammonium bromide, are exemplary of typical quaternary ammonium antimicrobial agents. Other compounds are bis [4-(R-amino)-1-pyridinium] alkanes as disclosed in U.S. Pat. No. 4,206,215 to Bailey. The pyridinium compounds are the preferred quaternary ammonium compounds, particularly preferred being cetylpyridinium, or tetradecylpyridinium halide salts (i.e., chloride, bromide, fluoride and iodide). Particularly preferred are cetylpyridinium chloride and fluoride salts.

The oral care composition can comprise at least about 0.025%, at least about 0.035%, at least about 0.045% to about 1.0%, from about 0.025% to about 1%, or from about 0.01% to about 10%, by weight of the composition, of the quaternary ammonium compound. Alternatively, the oral care composition can be essentially free of, substantially free of, or free of a quaternary ammonium compound.

pH

The pH of the oral care compositions as described herein can be from about 4 to about 10, from about 7 to about 10, greater than 7 to about 10, greater than 8 to about 10, greater than 7, greater than 7.5, greater than 8, greater than 9, from about 8.5 to about 10, from about 4 to about 7, from about 4 to about 6, from about 4.5 to about 6.5, from about 4.5 to about 5.5, from about 4 to less than 5.5, from about 4.5 to less than 5.5, greater than 4 to less than 5, greater than 4 to about 4.9, from about 4.9, from about 4 to about 5.4, from about 4 to about 5.3, from about 4 to about 5.2, from about 4 to about 5.1, from about 4 to about 5, from about 4 to about 4.9, from about 4 to about 4.8, from about 4 to about 4.7, or from about 4.8 to about 5.3. The pH of a mouth rinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

If the oral care composition comprises one or more dicarboxylic acids, a preferred pH is below about 7 or below about 6 due to the pKa of the dicarboxylic acid. While not wishing to be bound by theory, it is believed that the dicarboxylic acid displays unique behavior when the pH is below about 7 or below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential for demineralization is greatly increased. Consequently, the oral care compositions comprising dicarboxylic acid, as described herein, can preferably have a pH from about 4 to about 7, from about 4 to about 6, from about 4.5 to about 6.5, from about 4 to about 5, from about 4 to less than 5, from about 4 to about 4.9, or from about 4.5 to less than 5.5 to minimize metal hydroxide/metal oxide formation and any increased demineralization in the oral cavity.

The pH of the oral care composition, as described herein, can be measured either immediately upon mixing, or upon aging the composition by placing the oral care composition at ambient or accelerated temperature and humidity conditions, such as including measuring the pH at a temperature of 25° C., 30° C. and/or 40° C. with a 30%, 60% and/or 75% relative humidity for about 28 days or longer prior to measuring the pH.

### Buffering Agents

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred polyphosphates include linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Another suitable class of anionic surfactants are alkyl phosphates. The surface active organophosphate agents can have a strong affinity for enamel surface and have sufficient surface binding propensity to desorb pellicle proteins and remain affixed to enamel surfaces. Suitable examples of organophosphate compounds include mono-, di- or triesters represented by the general structure below:

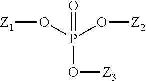

wherein Z₁, Z₂, or Z₃ may be identical or different with at least one being an organic moiety. Z₁, Z₂, or Z₃ can be selected from linear or branched, alkyl or alkenyl group of from 1 to 22 carbon atoms, optionally substituted by one or more phosphate groups; alkoxylated alkyl or alkenyl, (poly) saccharide, polyol or polyether group. Some other agents include alkyl or alkenyl phosphate esters represented by the following structure:

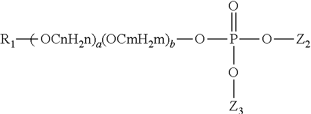

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z and Z may be identical or different, each represents hydrogen, alkali metal, ammonium, protonated alkyl amine or protonated functional alkylamine, such as analkanolamine, or a R—(OCH₂)(OCH)— group. Examples of suitable agents include alkyl and alkyl (poly)alkoxy phosphates such as lauryl phosphate; PPGS ceteareth-10 phosphate; laureth-1 phosphate; laureth-3 phosphate; laureth-9 phosphate; trilaureth-4 phosphate; C₁₂₋₁₈ PEG 9 phosphate; and sodium dilaureth-10 phosphate. The alkyl phosphate can be polymeric. Examples of polymeric alkyl phosphates include those containing repeating alkoxy groups as the polymeric portion, in particular 3 or more ethoxy, propoxy isopropoxy or butoxy groups.

Other suitable anionic surfactants are sarcosinates, isethionates and taurates, especially their alkali metal or ammonium salts. Examples include: lauroyl sarcosinate, myristoyl sarcosinate, palmitoyl sarcosinate, stearoyl sarcosinate oleoyl sarcosinate, or combinations thereof.

Other suitable anionic surfactants include sodium or potassium alkyl sulfates, such as sodium lauryl sulfate, acyl isethionates, acyl methyl isethionates, alkyl ether carboxylates, acyl alaninates, acyl gulatames, acyl glycinates, acyl sarconsinates, sodium methyl acyl taurates, sodium laureth sulfosuccinates, alpha olefin sulfonates, alkyl benze sulfonates, sodium lauroyl lactylate, sodium laurylglucosides hydroxypropyl sulfonate, and/or combinations.

A suitable taurate surfactant is represented by formula (I):

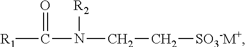

wherein R₁ is a saturated or unsaturated, straight, or branched alkyl chain with 6 to 18 C atoms; R₂ is H or methyl, and M is H, sodium, or potassium. Preferably, the R₁ is a saturated or unsaturated, straight, or branched alkyl chain with 8 to 18 C atoms. Optionally but preferably, the taurate surfactant comprises one or more selected from the group consisting of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, and combinations thereof.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio) acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocoamidoethyl betaine, cocoamidopropyl betaine (CADB), and lauramidopropyl betaine. Other suitable amphoteric surfactants include betaines, sultaines, sodium laurylamphoacetates, alkylamphodiacetates, and/or combinations thereof.

Suitable cationic surfactants include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Suitable nonionic surfactants include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The one or more surfactants can also include one or more natural and/or naturally derived surfactants. Natural surfactants can include surfactants that are derived from natural products and/or surfactants that are minimally or not processed. Natural surfactants can include hydrogenated, non-hydrogenated, or partially hydrogenated vegetable oils, olus oil, Passiflora incarnata oil, candelilla cera, coco-caprylate, caprate, dicaprylyl ether, lauryl alcohol, myristyl myristate, dicaprylyl ether, caprylic acid, caprylic ester, octyl decanoate, octyl octanoate, undecane, tridecane, decyl oleate, oleic acid decylester, cetyl palmitate, stearic acid, palmitic acid, glyceryl stearate, hydrogenated, non-hydrogenated, or partially hydrogenated vegetable glycerides, Polyglyceryl-2 dipolyhydroxystearate, cetearyl alcohol, sucrose polystearate, glycerin, octadodecanol, hydrolyzed, partially hydrolyzed, or non-hydrolyzed vegetable protein, hydrolyzed, partially hydrolyzed, or non-hydrolyzed wheat protein hydrolysate, polyglyceryl-3 diisostearate, glyceryl oleate, myristyl alcohol, cetyl alcohol, sodium cetearyl sulfate, cetearyl alcohol, glyceryl laurate, capric triglyceride, coco-glycerides, lectithin, dicaprylyl ether, xanthan gum, sodium coco-sulfate, ammonium lauryl sulfate, sodium cocoyl sulfate, sodium cocoyl glutamate, polyalkylglucosides, such as decyl glucoside, cetearyl glucoside, cetyl stearyl polyglucoside, coco-glucoside, and lauryl glucoside, and/or combinations thereof. Natural surfactants can include any of the Natrue ingredients marketed by BASF, such as, for example, CegeSoft®, Cetiol®, Cutina®, Dehymuls®, Emulgade®, Emulgin®, Eutanol®, Gluadin®, Lameform®, LameSoft®, Lanette®, Monomuls®, Myritol®, Plantacare®, Plantaquat®, Platasil®, Rheocare®, Sulfopon®, Texapon®, and/or combinations thereof.

Other specific examples of surfactants include sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Additional surfactants desired include fatty acid salts of glutamate, alkyl glucoside, salts of taurates, betaines, caprylates, and/or mixtures thereof. The oral care composition can also be sulfate free. The oral care composition can comprise one or more surfactants each at a level from about 0.01% to about 15%, from about 0.3% to about 10%, or from about 0.3% to about 2.5%, by weight of the oral care composition.

### Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e., with two functional groups), tridentate (i.e., with three functional groups), tetradentate (i.e., with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, citric acid, phytic acid, pyrophosphate, tripolyphosphate, tetrapolyphosphate, hexametaphosphate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

### Thickening Agent

The oral care composition can comprise one or more thickening agents. Thickening agents can be useful in the oral care compositions to provide a gelatinous structure that stabilizes the composition against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Vecgum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of embodiments of the present invention can comprise an abrasive. Abrasives can be added to oral care formulations to help remove surface stains from teeth. The oral care can include a calcium abrasive and/or a non-calcium abrasive, such as a silica abrasive.

The oral care composition can comprise a calcium abrasive. The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can comprise a non-calcium abrasive such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise from about 5% to about 70%, from about 10% to about 50%, from about 10% to about 60%, from about 20% to about 50%, from about 25% to about 40%, or from about 1% to about 50%, by weight of the oral care composition, of the non-calcium abrasive.

Alternatively, the oral care composition can be essentially free of, substantially free of, essentially free of, or free of silica, alumina, or any other non-calcium abrasive. The oral care composition can comprise less than about 5%, less than about 1%, less than about 0.5%, less than about 0.1%, or 0% of a non-calcium abrasive, such as silica and/or alumina.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

Where the oral care composition comprises a dicarboxylic acid, the oral care composition can include a low level of or no abrasive as the dicarboxylic acid can provide a high enough whitening benefit that an abrasive is not necessary.

While mouth rinse compositions typically do not include abrasive, dentifrice compositions typically do include abrasive. However, the dentifrice compositions and/or toothpaste compositions of embodiments of the present invention can include a low level of or no abrasive. As such, the oral care composition or dentifrice composition can comprise less than about 5%, from about 0.5% to about 2%, or less than about 2%, by weight of the composition, of abrasive. The oral care composition or dentifrice composition can also be essentially free of, substantially free of, or free of abrasive.

### Prenylated Flavonoids

The oral care composition can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula IX), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

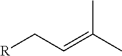

Formula IX. Prenyl Function Group with R representing the other portions of the molecule

Other suitable functionalities of the parent molecule that improve the structure-activity relationship (e.g., structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula X (flavone backbone), Formula XI (isoflavan backbone), and/or Formula XII (neoflavonoid backbone).

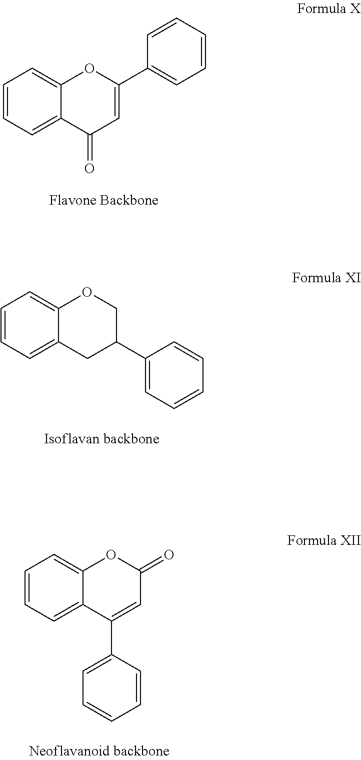 [SMILES: O=c1cc(-c2ccccc2)oc2ccccc12 ; c1ccc(C2COc3ccccc3C2)cc1 ; O=c1cc(-c2ccccc2)c2ccccc2o1]

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin A1, Epimedin B, Epimedin C, Icariin, Icariside I, Icariside II, Icaritin, Isobavachalcone, Isoxanthohumol, Neobavaisoflavone, 6-Prenylnaringenin, 8-Prenylnaringenin, Sophoraflavanone G, (−)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowighteone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having a MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (−)-Sophoranone, Kurarinone, Kuwanon C, Panduratin A, and/or combinations thereof.

Preferably, the prenylated flavonoid has a high probability of having a MIC of less than about 25 ppm for E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosae and Moraceae, due to their consumption as fruits and vegetables, are the most frequently investigated families and many novel prenylated flavonoids have been explored. Humulus lupulus of the Cannabaceae include 8-prenylnaringenin and xanthohumol, which can play a role in the health benefits of beer.

The prenylated flavonoid can be incorporated through a hops extract, incorporated in a separately added extract, or added as a separate component of the oral care compositions disclosed herein.

Suitable prenylated flavonoids can have a particular octanol-water partitioning coefficient. The octanol-water partitioning coefficient can be used to predict the lipophilicity of a compound. Without wishing to being bound by theory, it is believed that compounds that fall within the ranges described herein will be able to enter and/or disrupt the primarily hydrophobic phospholipid bilayer that makes of the cell membrane of microorganisms. Thus, the octanol-water partitioning coefficient can be correlated to the antibacterial effect of prenylated flavonoids. Suitable prenylated flavonoids can have a log P of at least about 2, at least about 4, from about 2 to about 10, from about 4 to about 10, from about 4 to about 7, or from about 4 to about 7.

The oral care composition can comprise at least about 0.001%, from about 0.001% to about 5%, from about 0.01% to about 2%, from about 0.0001% to about 2%, or at least about 0.05% of prenylated flavonoid.

### Amino Acid

The oral care composition can comprise amino acid. The amino acid can comprise one or more amino acids, peptide, and/or polypeptide, as described herein.

Amino acids, as in Formula XIII, are organic compounds that contain an amine functional group, a carboxyl functional group, and a side chain (R in Formula XIII) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula XIII, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

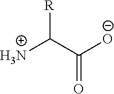

Formula XIII. Amino Acid. R is any suitable functional group

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in embodiments of the present invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, the neutral amino acids used in embodiments of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e., uncomplexed). If the neutral amino acid is in its salt form, suitable salts include salts known in the art to be pharmaceutically acceptable salts considered to be physiologically acceptable in the amounts and concentrations provided. Preferably the neutral amino acid is present in the amount of from about 0.0001% to about 10%, preferably from about 0.05% to about 5%, preferably from about 0.1% to about 3%, preferably from about 0.5% to about 3%, preferably from about 1% to about 3%, by weight of the composition. In one aspect, the neutral amino acid is glutamine (or salt thereof). In another aspect, the neutral amino acid is proline (or salt thereof). In yet another aspect, the neutral amino acid is glycine (or salt thereof).

The oral care composition can comprise from about 0.0001% to about 20%, from about 0.1% to about 10%, from about 0.5% to about 6%, or from about 1% to about 10% of neutral amino acid, by weight of the oral care composition.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

### Humectant

The oral care composition can comprise one or more humectants, have low levels of a humectant, be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition according to embodiments of the present invention can be anhydrous, a low water formulation, or a high water formulation. In total, the oral care composition can comprise from 0% to about 99%, from about 5% to about 75%, about 20% or greater, about 30% or greater, about 50% or greater, up to about 45%, or up to about 75%, by weight of the composition, of water.

In a high water oral care composition and/or toothpaste formulation, the oral care composition comprises from about 45% to about 75%, by weight of the composition, of water. The high water oral care composition and/or toothpaste formulation can comprise from about 45% to about 65%, from about 45% to about 55%, or from about 46% to about 54%, by weight of the composition, of water. The water may be added to the high water formulation and/or may come into the composition from the inclusion of other ingredients.

In a low water oral care composition and/or toothpaste formulation, the oral care composition comprises from about 5% to about 45%, by weight of the composition, of water. The low water oral care composition can comprise from about 5% to about 35%, from about 10% to about 25%, or from about 20% to about 25%, by weight of the composition, of water. The water may be added to the low water formulation and/or may come into the composition from the inclusion of other ingredients.

In an anhydrous oral care composition and/or toothpaste formulation, the oral care composition comprises less than about 10%, by weight of the composition, of water. The anhydrous composition comprises less than about 5%, less than about 1%, or 0%, by weight of the composition, of water. The water may be added to the anhydrous formulation and/or may come into the composition from the inclusion of other ingredients.

The oral care composition can also be a mouth rinse formulation. A mouth rinse formulation can comprise from about 75% to about 99%, from about 75% to about 95%, or from about 80% to about 95% of water.

The dentifrice composition can also comprise other orally acceptable carrier materials, such as alcohol, humectants, polymers, surfactants, and acceptance improving agents, such as flavoring, sweetening, coloring and/or cooling agents.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, cucalyptol, cassia, 1-menthyl acetate, sage, eugenol, parsley oil, oxanone, alpha-irisone, marjoram, lemon, orange, propenyl guacthol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions forms include emulsion compositions, such as the emulsions compositions of U.S. Pat. No. 11,147,753, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, such as the jammed oil-in-water emulsions of U.S. Pat. No. 11,096,874, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care compositions, as described herein, can lead to oral health benefits, such as the treatment, reduction, and/or prevention of caries, cavities, gingivitis, and/or combinations thereof and/or the whitening of teeth, removing stain from teeth, and/or preventing the accumulation of stain from teeth when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure.

The user can also be instructed to rinse with a mouthwash and/or mouth rinse composition after the completion of the brush procedure or instead of the brush procedure. The user can be instructed to swish the oral care composition thoroughly for at least 30 seconds, at least one minute, at least 90 seconds, or at least two minutes at least once, at least twice, or at least three times per day. The user can also be instructed to expectorate the oral care composition after the completion of the procedure.

The oral care compositions according to embodiments of the present invention can be used in the treatment, reduction, and/or prevention of caries, cavities, gingivitis, and/or combinations thereof. The oral care compositions according to embodiments of the present invention can be used to provide a whitening benefit, such as the whitening of teeth, removing stain from teeth, and/or preventing the accumulation of stain from teeth. For example, as described herein, hops beta acid can be useful as an antigingivitis agent. Thus, the addition of hops to any oral care composition can provide antigingivitis protection.

The oral care composition can include primary packaging, such as a tube, bottle, and/or tub. The primary package can be placed within secondary package, such as a carton, shrink wrap, or the like. Instructions for use of the oral care composition can be printed on the primary package and/or the secondary package. The scope of the method is intended to include instructions provided by a manufacturer, distributor, and/or producer of the oral care composition.

If the oral care composition is a toothpaste, the user can be instructed to dispense the toothpaste from the toothpaste tube.

The user can be instructed to apply a portion of the toothpaste onto a toothbrush. The portion of the toothpaste can be of any suitable shape, such as strip, a pea-sized amount, or various other shapes that would fit onto any mechanical and/or manual brush head. The user can be instructed to apply a strip of the toothpaste that is at least about 1 inch, at least about 0.5 inch, at least 1 inch, and/or at least 0.5 inch long to the bristles of a toothbrush, such as soft-bristled toothbrush.

The user can be instructed to apply pea-sized or grain of rice-sized portion of the toothpaste to the bristles of a toothbrush, such as in the case of use by children of less than 6 years old and/or less than 2 years old.

The user can be instructed to brush their teeth for at least about 30 seconds, at least about 1 minute, at least about 90 seconds, at least about 2 minutes, at least 30 seconds, at least 1 minute, at least 90 seconds, and/or at least 2 minutes.

The user can be instructed to brush their teeth thoroughly and/or as directed by a physician and/or dentist.

The user can be instructed to brush their teeth after each meal. The user can be instructed to brush their teeth at least once per day, at least twice per day, and/or at least three times per day. The user can be instructed to brush their teeth no more than three times a day, such as to prevent Sn staining. The user can be instructed to brush their teeth in the morning and/or in the evening prior to sleeping.

The user can be instructed to not swallow the toothpaste composition due to the inclusion of ingredients that are not suitable for ingestion, such as fluoride. However, in the case of an oral care composition comprising hops, but free of fluoride, the user may not need to be instructed to not swallow the toothpaste. The user may be instructed to expectorate (or spit out) the toothpaste composition after the cessation of the brushing cycle.

If the oral care composition is a mouth rinse, the user can be instructed to dispense the mouth rinse from a bottle containing the mouth rinse.

The user can be instructed to use the mouth rinse at least once a day, at least twice a day, and/or at least three times a day.

The user can be instructed to use the mouth rinse composition after the use of toothpaste and/or floss.

The user can be instructed to swish a portion of rinse in the oral cavity, such as between the teeth, for a period of time. The user can be instructed to vigorously swish a portion of the rinse.

The user can be instructed to use be from about 5 mL to about 50 mL, from about 10 mL to about 40 mL, 10 mL, 20 mL, 25 mL, 30 mL, 40 mL, 2 teaspoonfuls, and/or 4 teaspoonfuls of mouth rinse.

The user can be instructed to swish the mouth rinse for at least about 30 seconds, at least about 1 minute, at least about 90 seconds, at least about 2 minutes, at least 30 seconds, at least 1 minute, at least 90 seconds, and/or at least 2 minutes.

The user can be instructed to not swallow the mouth rinse composition due to the inclusion of ingredients that are not suitable for ingestion, such as fluoride. However, in the case of an oral care composition comprising hops, but free of fluoride, the user may not need to be instructed to not swallow the mouth rinse. The user may be instructed to expectorate (or spit out) the mouth rinse composition after the cessation of the rinse cycle.

The usage instructions for the oral care composition, such as for a toothpaste composition and/or a mouth rinse composition, can vary based on age. For example, adults and children that are at least 6 or at least 2 can have one usage instruction while children under 6 or under 2 can have a second usage instruction.

The oral care composition, as described herein, can be useful as medicament, such as in an anticavity and/or antigingivitis treatment, as described herein. Suitable medicaments include oral care compositions, toothpaste compositions, mouth rinse compositions, floss coatings, chewing gums, and/or other suitable compositions to be applied in the oral cavity.

Additionally, the oral care composition, as described herein, can be used to reduce the number and/or intensity of white spots on teeth, which can be attributable to caries presence within the oral cavity. Or the oral care composition, as described herein, can be used to reduce the redness, puffiness, tenderness, and/or swollenness of gums at the gumline immediately adjacent the surfaces of the teeth, which can be attributable to gingivitis presence within the oral cavity.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Fluoride Uptake

The Enamel Fluoride Uptake by FDA Method #40 is a method used to determine the amount of fluoride delivered to demineralized enamel specimens from a single, 30-minute treatment of 1:3 dentifrice slurry supernatant.

Cores of sound human enamel with diameters of 3-4 mm were extracted from whole human teeth. The cores were mounted on acrylic rods and the surfaces were ground using 600 grit wet/dry sandpaper. The cores were then polished with 0.05μ polish (Alumina Suspension Gamma B, MetLab Corp, catalog #M303-128) to a mirror finish. Specimens were stored in an airtight container above a small amount of deionized water (about 1-5 mL) in a standard laboratory refrigerator (about 2-4° C.).

Each enamel specimen was inspected and samples with large cracks or uneven calcification were discarded. Specimens were polished again for 10 minutes using 0.05μ polish. Samples were sonicated with a sonicator in deionized water for 15-30 min. Enamel specimens were then rinsed with standard deionized water and wiped to remove any residual polish.

Enamel specimens were then demineralized. For each specimen, 25 mL of MHDP (N-2-hydroxyethyl, methane hydroxy diphosphonate) demineralization solution (0.025M lactic acid, 2×10⁻⁴ M MHDP) was placed in a 30 mL plastic vial. An enamel specimen was placed through the cap of each vial. Each cap was placed on the top of the vial to submerge the enamel specimen in the MHDP demineralization solution. The enamel specimen was not allowed to touch the bottom of the vial. Specimens were left in the demineralization solution for 48 hours at ambient conditions to form artificial caries lesions. The rods were tapped twice daily to remove any bubbles. After 48 hours, specimens were removed from the demineralization solution and rinsed thoroughly with deionized water.

If the sample was a paste dentifrice, 10 g of dentifrice was placed in a 50 mL tri-pour plastic beaker. 30 mL of deionized water was added to the beaker. An x-shaped stir bar was placed on top of the dentifrice in each beaker and the beaker was placed on a magnetic stir plate. The dentifrice was broken up with a wooden stick until the stir bar was capable of spinning freely at 300-400 rpm. The dentifrice slurry was stirred for 20 minutes. The slurry was transferred to a centrifuge tube and centrifuged for 30 minutes at 11,000 rpm.

Slurry supernatants were decanted into a 50 mL tri-pour plastic beaker. An x-shaped stir bar was placed in the beaker and the beaker was placed on a magnetic stir plate. The stir plate was turned to 300-400 rpm. Lesioned enamel specimens were suspended into each treatment. Each sample was treated for 30 minutes. After 30 minutes, each sample was rinsed with deionized water. Samples were stored in an airtight container above a small amount of deionized water (about 1-5 mL) in a standard laboratory refrigerator (about 2-4° C.).

The samples were analyzed for fluoride content analysis by collecting a portion of milled enamel powder following drilling to a depth of 50 micro-meters, dissolving that enamel in acid, then neutralizing and buffering it. Upon drilling a sample from the enamel specimen, the area of the enamel drilled was recorded.

Fluoride uptake was directly measured using a Fluoride Ion Specific Electrode (Thermo Scientific, Orion, 96-09-00, Waltham, MA, USA). Each specimen sample was placed on the end of the electrode. A value of mV was recorded. This value was converted to ppm fluoride by using a standard curve of prepared fluoride standards. Fluoride uptake was calculated by dividing the mass of fluoride in μg by the total area sampled with the microdrill biopsy.

### HAP Dissolution

The HAP dissolution method was designed to test the acid protection of a chosen test oral care composition. After treating hydroxyapatite (HAP) powder with test diluted solutions, the HAP was added to an acidic media, and the change in pH was an indicator of the degree of surface protection from acid.

Diluted solutions (1:3 concentrate solution: water) were prepared for all treatment compositions. Specifically, 10 g of oral care composition was combined with 30 g of deionized, ultra-pure water in a 50 mL container with a stir bar. If a dentifrice was used, 10 g the dentifrice was broken up with a spatula until the stir bar moved freely at 300-400 rpm. The slurry was mixed on the stir plate for about 10 to 20 minutes and/or until a uniform slurry was formed. The paste slurries were centrifuged at 15,000 rpm for 15 min to separate the solid components from the supernatant. If solutions were used, they were used without this centrifugation step.

For each treatment, including for the water control, 0.300 g of hydroxyapatite powder was placed into a 50 mL round bottom centrifuge tube with 4, 4 mm glass beads. For treatment with an oral care composition, 24 mL of the prepared dentifrice supernatant or diluted solution was added to the HAP. Each treated HAP sample was immediately vortex mixed at 2500 rpm for 2 minutes. All samples were then centrifuged at 15,000 rpm for 15 minutes. The liquid phase was decanted out of the centrifuge tube, which left a HAP pellet and glass beads. The remaining HAP pellet was rinsed by adding deionized water, vortex mixing at 2500 rpm for 1 minute to completely disperse the pellet, centrifuging at 15,000 rpm for 15 minutes, and the liquid phase was decanted out of the centrifuge tube then discarded. This rinsing step was repeated two more times. The treated HAP pellet was dried in a 55° C. oven overnight.

Samples of HAP were analyzed for change in pH (ΔpH). 25 mL of 10 mM citric acid (1.9212 g of citric acid in 1 L of deionized water) was added to a 50 ml beaker with a stir bar. The beaker was placed on a stir plate (Metrohm, Herisau, Switzerland, Model No. 728) and turned on. A Titrano pH electrode (Metrohm, Herisau, Switzerland, Model No. 719S) was placed in the stirring beaker with citric acid. After equilibration of the citric acid solution (until pH reads 2.5±0.001 pH for 30 seconds), 50 mg of the dried HAP powder was added to the citric acid solution. The pH was monitored and the value was recorded at 5 minutes. The ΔpH was determined by subtracting the pH reading at 5 minutes from the stable pH reading obtained immediately prior to adding the treated HAP powder. The results for HAP solubility reduction are provided herein.

Example oral care compositions were made by dissolving the necessary carboxylic acid(s) in 90 mL of ultra-pure water in a glass beaker using a stir bar and magnetic stir plate. After the acid(s) were completely dissolved, stannous fluoride (0.454 g per 100 g solution) was added and stirred until completely dissolved. The solution was then neutralized dropwise with IN NaOH dropwise until pH 4.5 was reached. The solution was transferred to a 100 mL Erlenmeyer flask and ultra-pure water was added to reach 100 mL. The solutions where then tightly stoppered and stored in a refrigerator until use. To treat the HAP powder, a quantity of solution was diluted 1 part solution (10 g) and 3 parts ultra-pure water (30 g) as if it were a toothpaste. The HAP powder was then treated as normal according to the method above.

### Chemical Pellicle Cleaning Ratio (C-PCR)

The method of the Chemical Pellicle Cleaning Ratio (C-PCR) is similar to that of the Pellicle Cleaning Ratio (PCR), which is a well-accepted industry method to investigate the extrinsic stain removal properties of abrasive-containing oral care compositions or toothpastes as a means to estimate their clinical stain removal potential. The PCR method was originally published by Stookey et al. (1982) and was later refined by Schemehorn et al. (2011) to make a darker, more tenacious stain. The stained tooth chips from the method of Schemehorn et al. were used here to evaluate the ability of the dicarboxylate-containing formulations to remove a dental stain mimic. In the C-PCR method, instead of brushing the chips, they are tumbled overnight (16 hours). Four chips are used per 50 mL Falcon tubes with 40 mL of toothpaste slurry. Two tubes are used to collect data for the eight chips that constitute a treatment group. The pre- and post-tumbling L* values were used to determine the reduction in tooth stain from the 16 hours of chemistry exposure as measured by ΔL*. The statistical grouping was determined using a difference test with α=0.05 using the JMP statistical software package. Treatments with different letter codes are statistically significantly different, p<0.05.

### Soluble Sn

This method is suitable for determination of soluble tin in oral care toothpaste or dentifrice compositions from about 5 to about 5,000 ppm Sn in the aqueous slurry supernatant. The slurry was prepared by mixing 1 part toothpaste with 3 parts water. An aliquot of slurry was acid digested, diluted, and analyzed by inductively coupled plasma optical emission spectrometry (ICP-OES) for each toothpaste measured. Results are reported here as ppm in the neat aqueous phase of the toothpaste and/or dentifrice.

Several standards and reagents were prepared prior to the beginning of the analysis. A 5% hydrochloric acid/5% Nitric acid rinse solution was prepared by transferring 100 mL each of concentrated HCl and concentrated HNO₃ using a graduated cylinder to a 2 L volumetric flask containing about 1 L of ultrapure, 18 MΩ (DI) water. The solution was swirled to mix and diluted to the mark of the graduated flask then mixed well by repeated flask inversion.

A 1000 mg/L tin and 1000 mg/L gallium standard solution were purchased (Sigma Aldrich, Merck KGaA, Darmstadt, Germany) for preparation of the standard solutions according to Table 1. A pipet was used to transfer accurate quantities of the standards to a 50 mL volumetric flask while a graduated cylinder was used for the concentrated acids. After transfer, the volumetric flask was filled to the line with DI water and mixed well.

TABLE 1
Soluble Sn Standard Solution Compositions

| | | | 1000 mg/L | 1000 mg/L |
|---|---|---|---|---|
| | Conc HNO₃ | Conc HCL | Sn Std | Ga Std |
| Solution | (mL) | (mL) | (mL) | (mL) |
| Cal Blank | 2.5 | 2.5 | 0 | 0.2 |
| Cal 10 mg/L Sn | 2.5 | 2.5 | 0.5 | 0.2 |
| LLOQ 0.5 mg/L | 2.5 | 2.5 | 0.025 | 0.2 |
| Sn | | | | |
| QC 5 mg/L Sn | 2.5 | 2.5 | 0.25 | 0.2 |

Slurries were prepared by weighing 2.00 grams of sample into a tared round bottom 38 mL centrifuge tube containing 10 glass beads. The weight was recorded to a minimum of 0.001 g. Immediately before slurrying, 6.0 mL of DI water was transferred to the tubes. Tubes were capped and placed on a vortexer, mixing the samples for 60 minutes at 1200 rpm. The tubes were removed from the vortexer immediately following completion of the mixing cycle and placed in a centrifuge. They were centrifuged at 21,000 relative centrifugal force (RCF) for 10 minutes. Immediately following completion of centrifugation, the tubes were removed, and the supernatant was gently mixed by inverting slowly three times making sure the solid plug at the bottom of the centrifuge tube was not disturbed before the sample was decanted. The supernatant was then decanted into a 15 mL screw cap sample tube, making sure most of the supernatant was transferred.

The supernatant samples were then digested by accurately weighing (to 0.001 g) a 0.5 mL aliquot of supernatant into a 50 mL Falcon tube. Then 2.5 mL of concentrated HCl and HNO₃ were added. The tubes were covered with a polypropylene watch glass and placed in a preheated block digester at 90° C. for 30 minutes. The samples were removed the from the heat, the watch class was rinsed three times with DI water (with about 1 mL each time), and that rinsate was added to the digested supernatant. The gallium standard (0.2 mL) was pipetted into the digested supernatant and then the supernatant samples were diluted to 50 mL with DI water. The tubes were capped and mixed. A digestion method blank was prepared in the same manner using 0.5 mL of DI water instead of supernatant. A method blank was prepared and analyzed for each set of hot block digestions if more samples were prepared than could fit into the hot block at once.

The ICP-OES (Perkin-Elmer 8300, Waltham, MA, USA) was operated by a trained and qualified operator with demonstrated capability of running the instrument and accurately determining the quantity of tin in oral care compositions. The ICP-OES operation parameters were selected based on the model and configuration according to the manufacturer's instructions. Samples were analyzed according to the following protocol:
1. The ICP-OES was preheated and optimized according to the manufacturer's guidelines. Recommended system checks were performed. The system was conditioned for 30 minutes prior to analysis by running the HCl/HNO₃ rinse solution through the sample introduction system.
2. The method for determining tin using a gallium internal standard at the manufacturer recommended wavelengths, integration times, and observation modes was loaded into the operating computer.
3. The 5% HCl/5% HNO₃ rinse solution was used to rinse the sample introduction system between the analysis of each blank, standard, or test solution.
4. Three to five readings were recorded for all solutions during analysis.
5. The calibration blank was analyzed.
6. The 10 ppm Sn standard was measured.
7. The 5 ppm Sn standard was measured.
8. The 0.5 ppm LLOQ tin standard was measured.
9. The method blank was measured.
10. The test solutions were measured.
11. The 5 ppm Sn standard was re-measured after every sixth test solution and after the last sample. Enough standard was made to complete the analysis.
12. The 0.5 ppm LLOQ tin standard was measured at the end of the sample analysis. The analysis was considered successful if the % relative standard deviation of the replicate readings for the 10 ppm and the 5 ppm tin standards was less than about 3%. The 5-ppm check standard was within 96-104% of its value. The LLOQ was within 75-125% of its value. The method blank showed less tin signal intensity than the LLOQ sample. The recovery of the internal standard in each analyzed solution was within 90-130% of its value.

The soluble tin was determined according to the following formula:

$$FORMULA IV$$

$$Soluble Tin in Composition = \frac{Sn from ICP ( \frac{μg}{mL} ) \times Final volume of test solution ( mL )}{Supernatant Weight ( g )} \times \frac{Soluble Mass of Composition ( g ) + Slurry Water ( g )}{Total Composition Mass ( g )} .$$

### Oral Care Compositions

The oral care composition of Tables 2 and 3 were prepared by combining one or more humectants, water, sweetener(s), and whitening agents to create a liquid mixture. The liquid mixture was homogenized at 25° C. until homogeneous and completely dissolved. Next, sodium hydroxide (50% solution) was added to the liquid mixture, and the liquid mixture was homogenized at 25° C. until homogeneous and completely dissolved. A separate powder mixture was prepared by combining the abrasive silica, thickening silica, and opacifier, with any thickening agents, such as xanthan gum and/or sodium carboxymethylcellulose. The powder mixture was then combined with the liquid mixture and homogenized completely. Next, the surfactant, such as sodium lauryl sulfate, and flavor were added to the mixture. The contents were homogenized at 25° C. until homogeneous and entrained air was removed by vacuum.

TABLE 2
Oral Care Compositions

| | Summary | | | |
|---|---|---|---|---|
| | | | | SnF₂ + |
| | | | SnF₂ + | SnCl₂ + |
| | | SnF₂ + | SnCl₂ + | gluc + |
| | SnF₂ + | SnCl2 + | gluc + | EDTA + |
| | EDTA | EDTA + Ox | EDTA + Ox | Mal |
| | pH 4.5 | pH 4.5 | pH 4.5 | pH 4.5 |
| Ingredient (wt %) | Ex. 1 | Ex. 2 | Ex. 3 | Ex. 4 |
| Sorbitol | 43.000 | 38.000 | 36.000 | 37.000 |
| Glycerin | 10.000 | 10.000 | 10.000 | 10.000 |
| Treated Water | 13.616 | 14.944 | 15.719 | 14.234 |
| SnF₂ | 0.454 | 0.454 | 0.454 | 0.454 |
| SnCl₂ (10% Silica | | 0.562 | 0.562 | 0.562 |
| Blend) | | | | |
| Sodium Gluconate | | | 1.300 | 1.300 |
| NaOH (50% | 0.105 | 0.075 | 0.000 | 1.850 |
| Solution) | | | | |
| Saccharin | 0.500 | 0.500 | 0.500 | 0.500 |
| Xanthan Gum | 0.350 | 0.350 | 0.350 | 0.350 |
| Carrageenan | 0.700 | 0.700 | 0.700 | 0.700 |
| Sodium Citrate | | | | |
| EDTA disodium salt | 2.600 | 2.600 | 2.600 | 2.600 |
| (dihydrate) | | | | |
| DTPA (anhydrous) | | | | |
| (>98%) | | | | |
| HEDP (60% | | | | |
| solution) | | | | |
| Potassium oxalate | | 3.140 | 3.140 | |
| (monohydrate) | | | | |
| Malonic acid | | | | 1.775 |
| TiO₂ | 0.500 | 0.500 | 0.500 | 0.500 |
| Silica | 22.000 | 22.000 | 22.000 | 22.000 |
| SLSS (29% | 5.000 | 5.000 | 5.000 | 5.000 |
| Solution) | | | | |
| Flavor | 1.175 | 1.175 | 1.175 | 1.175 |
| Total | 100 | 100 | 100 | 100 |
| Sn:Mono:Poly Ratio | 1:0:2.4 | 1:0:4.7 | 1:1.2:4.7 | 1:1.2:4.7 |
| Target pH | 4.5 | 4.5 | 4.5 | 4.5 |
| 1:3 Paste:Water | 4.87 | 4.72 | 4.57 | 4.58 |
| Slurry pH | | | | |

TABLE 3
Oral Care Compositions

| | | | SnF₂ + | | | |
|---|---|---|---|---|---|---|
| | | | SnCl₂ + | SnF₂ + | | |
| | | SnF₂ + | gluc + | SnCl₂ + | | |
| | SnF₂ + | SnCl₂ + | citrate + | gluc + | SnF₂ + | SnF₂ + |
| | EDTA | EDTA | EDTA | EDTA | DTPA | HEDP |
| | pH 6.5 | pH 6.5 | pH 6.5 | pH 6.5 | pH 6.5 | pH 6.5 |
| Ingredient (wt %) | Ex. 5 | Ex. 6 | Ex. 7 | Ex. 8 | Ex. 9 | Ex. 10 |
| Sorbitol | 43.000 | 42.000 | 38.000 | 40.000 | 40.000 | 42.000 |
| Glycerin | 10.000 | 10.000 | 10.000 | 10.000 | 10.000 | 10.000 |
| Treated Water | 13.006 | 13.164 | 14.409 | 13.809 | 14.414 | 12.931 |
| SnF₂ | 0.454 | 0.454 | 0.454 | 0.454 | 0.454 | 0.454 |
| SnCl₂ (10% Silica Blend) | | 0.562 | 0.562 | 0.562 | | |
| Sodium Gluconate | | | 1.300 | 1.300 | | |
| NaOH (50% soln) | 0.715 | 0.995 | 0.900 | 1.050 | 2.158 | 2.000 |
| Saccharin | 0.500 | 0.500 | 0.500 | 0.500 | 0.500 | 0.500 |
| Xanthan Gum | 0.350 | 0.350 | 0.350 | 0.350 | 0.350 | 0.350 |
| Carrageenan | 0.700 | 0.700 | 0.700 | 0.700 | 0.700 | 0.700 |
| Sodium Citrate | | | 1.550 | | | |
| EDTA disodium salt | 2.600 | 2.600 | 2.600 | 2.600 | | |
| (dihydrate) | | | | | | |
| DTPA (anhydrous) (>98%) | | | | | 2.750 | |
| HEDP (60% soln) | | | | | | 2.390 |
| Potassium oxalate | | | | | | |
| (monohydrate) | | | | | | |
| Malonic acid | | | | | | |
| TiO₂ | 0.500 | 0.500 | 0.500 | 0.500 | 0.500 | 0.500 |
| Silica | 22.000 | 22.000 | 22.000 | 22.000 | 22.000 | 22.000 |
| SLSS (29% soln) | 5.000 | 5.000 | 5.000 | 5.000 | 5.000 | 5.000 |
| Flavor | 1.175 | 1.175 | 1.175 | 1.175 | 1.175 | 1.175 |
| Total | 100 | 100 | 100 | 100 | 100 | 100 |
| Sn:Mono:Poly Ratio | 1:0:2.4 | 1:0:1.4 | 1:1.2:2.4 | 1:1.2:1.4 | 1:0:2.4 | 1:0:2.4 |
| Target pH | 6.5 | 6.5 | 6.5 | 6.5 | 6.5 | 6.5 |
| 1:3 Paste:Water Slurry pH | 6.74 | 6.81 | 6.61 | 6.79 | 6.96 | 6.98 |

Table 4 describes various comparative examples, which are all commercial dentifrice compositions.

TABLE 4
Comparative Toothpaste Composition

| | Crest ® Cavity | Crest ® | |
|---|---|---|---|
| | Protection | ProHealth | |
| Crest ® Cavity | (Fluoride | Advanced Deep | Colgate Cavity |
| Protection | Placebo) | Clean Mint | Protection |
| Sorbitol | Sorbitol | Glycerin | Dibasic Calcium |
| | | | Phosphate |
| | | | Dihydrate |
| Water | Water | Silica | Water |
| Silica | Silica | Sodium Hexa- | Sorbitol |
| | | metaphosphate | |
| Sodium Lauryl | Sodium Lauryl | Propylene Glycol | Sodium Lauryl |
| Sulfate | Sulfate | | Sulfate |
| Trisodium | Trisodium | PEG-6 | Carboxymethyl |
| Phosphate | Phosphate | | Cellulose, |
| | | | Sodium |
| Sodium | Sodium | Water | Sodium |
| Phosphate | Phosphate | | Pyrophosphate |
| Cellulose Gum | Cellulose Gum | Zinc Lactate | Sodium Saccharin |
| Carbomer | Carbomer | Flavor | Sodium |
| | | | Monofluoro- |
| | | | phosphate |
| Sodium | Sodium | Sodium Lauryl | |
| Saccharin | Saccharin | Sulfate | |
| Titanium | Titanium | Sodium Saccharin | |
| Dioxide | Dioxide | | |
| Dye | Dye | Trisodium | |
| | | Phosphate | |
| Sodium Fluoride | | Xanthan Gum | |
| | | PVP | |
| | | Carrageenan | |
| | | Cocamidopropyl | |
| | | Betaine | |
| | | Blue Dye | |
| | | Stannous Fluoride | |

### Results

The results in Table 5 illustrate the fluoride uptake of various inventive or comparative examples to deliver fluoride to artificially demineralized human enamel.

TABLE 5
Fluoride Uptake Results

| | | Fluoride | | | | | | | | | | | |
|---|---|---|---|---|---|---|---|---|---|---|---|---|---|
| | | Uptake | | | | | | | | | | | |
| Treatment | N | (μg F/cm²) | Statistical Group* | | | | | | | | | | |
| Crest ® Cavity Protection | 4 | 2.62 | | | | | | | | | | | K |
| (F Placebo) | | | | | | | | | | | | | |
| USP SnF₂/Silica | 4 | 6.61 | | | | | | | | | | J | |
| Ex. 1\hat{ } | 4 | 15.59 | A | | | | | | | | | | |
| Ex. 2\hat{ } | 4 | 14.45 | | B | | | | | | | | | |
| Ex. 3\hat{ } | 4 | 8.72 | | | | | E | F | | | | | |
| Ex. 4\hat{ } | 4 | 13.53 | | | C | | | | | | | | |
| Ex. 5\hat{ } | 4 | 12.36 | | | | D | | | | | | | |
| Ex. 6\hat{ } | 4 | 8.64 | | | | | E | F | | | | | |
| Ex. 7\hat{ } | 4 | 8.07 | | | | | | F | G | | | | |
| Ex. 8\hat{ } | 4 | 9.37 | | | | | E | | | | | | |
| Ex. 9 | 4 | 6.55 | | | | | | | | | I | J | |
| Ex. 10 | 4 | 6.98 | | | | | | | | H | I | J | |

*Means for groups in homogeneous subsets, those that do not share a letter are significantly different (alpha = 0.05), and sample size = N. Student-Newman-Keuls method.
\hat{ }Indicates a composition with an improved level of fluoride uptake relative to positive control.

Crest Cavity Protection (F Placebo) does not contain fluoride; therefore, the fluoride uptake value represents the amount of fluoride inherent in the demineralized tissue. USP SnF₂/silica is used as the positive control and is an anticavity toothpaste with unstabilized stannous fluoride delivered at a slurry pH of 4.65. At this pH, the stannous can rapidly precipitate onto the tooth surface forming a Sn(OH)ₓ coating that slows fluoride uptake. When the stannous is effectively stabilized to reduce the speed of Sn precipitation onto the tooth surface, the fluoride uptake can be enhanced vs. the USP SnF₂/silica (positive control). With respect to enamel fluoride uptake, the compositions with EDTA, Examples 1-8, enhanced the results relative to the positive control. However, the compositions with DTPA or HEDP, Examples 9 and 10, respectively, did not enhance the results with respect to the positive control. Higher fluoride uptake means more fluoride delivered to the demineralized tissue, which is an important anticaries mechanism of fluoride.

The HAP dissolution rate was determined for various inventive or comparative examples. The results are shown in Table 6.

TABLE 6
HAP Dissolution Results

| Treatment | N | Δ pH | Statistical Group* | | | | | |
|---|---|---|---|---|---|---|---|---|
| Crest ® Cavity | 3 | 1.01 | A | | | | | |
| Protection | | | | | | | | |
| USP SnF2/Silica | 3 | 0.89 | | | | | E | |
| Crest ® ProHealth | 3 | 0.78 | | | | | | F |
| Advanced Deep | | | | | | | | |
| Clean Mint | | | | | | | | |
| Ex. 1 | 3 | 0.99 | A | B | C | D | | |
| Ex. 2\hat{ } | 3 | 0.90 | | | | D | E | |
| Ex. 3\hat{ } | 3 | 0.91 | | | | D | E | |
| Ex. 4 | 3 | 1.01 | A | B | | | | |
| Ex. 5\hat{ } | 3 | 0.92 | | | C | D | E | |
| Ex. 6\hat{ } | 3 | 0.95 | | B | C | D | E | |
| Ex. 7\hat{ } | 3 | 0.93 | | B | C | D | E | |
| Ex. 8\hat{ } | 3 | 0.93 | | B | C | D | E | |
| Ex. 9 | 3 | 0.99 | A | B | C | | | |
| Ex. 10\hat{ } | 3 | 0.92 | | | | D | E | |

*Means for groups in homogeneous subsets, those that do not share a letter are significantly different (alpha = 0.05), and sample size = N. Student-Newman-Keuls method.
\hat{ }Indicates a composition with an improved level of HAP dissolution relative to Crest ® Cavity Protection

The results in Table 6 illustrate the ability of different treatments to reduce the dissolution rate of hydroxyapatite when exposed to 1% citric acid at pH of about 2.0. When a treated powder is exposed to citric acid, it begins to dissolve, and the pH rises. The better protected the surface, the smaller the pH rise over five minutes of exposure. The pH rise for a fluoride-only treatment, e.g., Crest Cavity Protection, is 1.01 units. The pH rise for poorly stabilized SnF₂, e.g., USP SnF₂/silica, is 0.89 units. The pH rise for stabilized fluoride and stannous with additional sodium hexametaphosphate, e.g., Crest ProHealth Advanced Deep Clean Mint, is 0.78 units. Examples 2, 3, 5, 6, 7, 8, and 10 all delivered Sn to the tooth surface in a way that was effective at reducing the dissolution rate of hydroxyapatite with respect to Crest Cavity Protection and in a way that was not different than USP SnF₂/silica. Better protection in this model corresponds to better protection of dental enamel from dietary acids. However, Examples 1, 4, and 9 were unable to deliver Sn to the enamel to reduce to the dissolution rate of hydroxyapatite with respect to Crest Cavity Protection. Additionally, Examples 1, 4, and 9 all provided significantly worse protection than that provided by USP SnF₂/silica.

The Chemical Pellicle Cleaning Ratio (C-PCR) was determined for various inventive or comparative examples. The results are shown in Table 7.

TABLE 7
C-PCR Results

| Treatment | N | ΔL* | Statistical Group ¹ | | | |
|---|---|---|---|---|---|---|
| Colgate Cavity Protection | 8 | −1 | | | | D |
| Ex. 1\hat{ } | 8 | 22 | | | C | |
| Ex. 2\hat{ } | 8 | 29 | | B | | |
| Ex. 3\hat{ } | 8 | 28 | | B | | |
| Ex. 4\hat{ } | 8 | 32 | A | | | |
| Ex. 5 | 8 | 2 | | | | D |
| Ex. 6 | 8 | 1 | | | | D |
| Ex. 7 | 8 | 2 | | | | D |
| Ex. 8 | 8 | 1 | | | | D |
| Ex. 9 | 8 | 1 | | | | D |
| Ex. 10 | 8 | 1 | | | | D |

¹ Means for groups in homogeneous subsets, those that do not share a letter are significantly different (alpha = 0.05), and sample size = N. Tukey's HSD method.
\hat{ }Indicates a composition with significantly improved ΔL*

The results in Table 7 illustrate the ability of the compositions to dissolve stain from the tooth surface. When a stained tooth chip is exposed to a slurry of the composition for 16 hours with agitation of the slurry, the stain can be dissolved. Importantly, the comparative example without stain removal agents, e.g., Colgate Cavity Protection, is unable to remove stain from the tooth surface chemically after 16 hours of exposure at room temperature. However, Examples 1-4 demonstrated a significant amount of tooth stain removal. Examples 5-10 could not remove tooth stain. A distinguishing feature of Examples 1-4 is their target pH of 4.5 vs Examples 5-10 with a target pH of 6.5.

The Soluble Sn for the various inventive examples with respect to the product age (days following manufacture) are shown in Table 8.

TABLE 8
Soluble Sn Results (% Remaining vs.
Formulated) Product Age at 40° C.

| Treatment | 0 days | 30 days | 60 days | 90 days | 180 days |
|---|---|---|---|---|---|
| Ex. 1 | 99.2 | 81.4 | 86.0 | 83.2 | 88.5 |
| Ex. 2 | 94.7 | 74.2 | 80.4 | 79.2 | 81.7 |
| Ex. 3 | 95.9 | 73.0 | 81.0 | 79.2 | 82.3 |
| Ex. 4 | 98.4 | 77.3 | 83.5 | 84.1 | 89.7 |
| Ex. 5 | 84.5 | 60.9 | 66.2 | 66.8 | 62.4 |
| Ex. 6 | 85.4 | 66.8 | 67.4 | 71.1 | 70.5 |
| Ex. 7 | 83.5 | 68.1 | 74.2 | 70.5 | 70.5 |
| Ex. 8 | 84.8 | 66.2 | 72.4 | 69.3 | 73.6 |
| Ex. 9 | 77.2 | 57.4 | 66.2 | 57.5 | 56.6 |
| Ex. 10 | 90.2 | 78.2 | 78.4 | 79.2 | 83.4 |

The results in Table 8 illustrate the change in soluble Sn content reported as percentage of the formulated content remaining with respect to days from manufacture. In the lower pH examples (Ex. 1-4), protonation of the silanol groups reduces their reaction with stannous in comparison to the higher pH examples (Ex. 5-10), thereby improving Sn stability. The additional choice of Sn stabilizers can also impact the stability of Sn, as illustrated negatively for DTPA (Ex. 9) and positively for HEDP (Ex. 10) vs. EDTA (Ex. 6). Without wishing to be bound by theory, stannous fluoride toothpastes without silica-based abrasives that are packaged in a sealed, oxygen-barrier tube exhibit negligible loss (e.g., <5%) of Sn over the shelf-life of the product. Whereas toothpastes containing silica abrasive lose stannous over time because of a reaction of stannous ions with oxygen (e.g., silanol groups) on the dental silica surface. However, reactivity towards silica abrasive was not predictive of optimal formulation conditions for the simultaneous enhancement of fluoride uptake and hydroxyapatite surface protection.

Without wishing to be bound by theory, these compositions are attempting to simultaneously enhance fluoride uptake, protect a hydroxyapatite surface from citric acid attack, and optionally remove tooth stain. If just enhancing fluoride uptake and protecting hydroxyapatite are considered, the inventive composition Ex. 1 can enhance fluoride uptake but cannot protect the tooth surface from citric acid attack. This is because the Sn-ligand complex is too stable thus preventing the Sn from interacting with the tooth surface. The challenge is to destabilize the Sn-ligand complex enough to allow Sn to protect the tooth surface while preserving its ability to enhance fluoride uptake. This reduction of Sn-ligand stability was achieved here in several ways.

The first way the Sn-EDTA ligand at target pH 4.5 (Ex. 1) stability was reduced was through addition of other ligand compounds. In Ex. 2 and Ex. 3, the use of oxalate or oxalate/gluconate at composition pH of 4.5 destabilized the Sn-ligand sufficiently to allow simultaneous protection of the tooth surface and enhancement of fluoride uptake. In Ex. 4, the use of malonate instead of oxalate allowed for enhancement of fluoride uptake but not for protection of the hydroxyapatite surface. Therefore, the Sn-EDTA ligand was not sufficiently destabilized by the malonate in Ex. 4.

The second way the Sn-EDTA ligand at target pH 4.5 (Ex. 1) stability was reduced was through raising the pH. Raising the pH can make the Sn more reactive with oxygen containing compounds like water or hydroxyapatite. Ex. 5 is identical to Ex. 1 but at target pH of 6.5. This was sufficient to destabilize the Sn-EDTA ligand and allow it to simultaneously enhance fluoride uptake and protect hydroxyapatite from acid attack. This approach worked when additional stannous was added (Ex. 6) and the ratio of Sn to Ligand was reduced. However, the compositions were unable to remove stain.

The third way the Sn-EDTA ligand at target pH 4.5 (Ex. 1) stability was reduced was through raising the pH and adding additional ligands that could compete with EDTA for the Sn and potentially reduce strength of the Sn-EDTA interaction. This worked when gluconate (Ex. 8) or gluconate and citrate (Ex. 7) were used. However, the compositions were unable to remove stain.

The fourth way was to fundamentally change the ligand to DTPA (Ex. 9) or HEDP (Ex. 10). Neither of these two ligands were able to achieve the simultaneous enhancement of fluoride uptake and protection of the hydroxyapatite surface from acid attack.

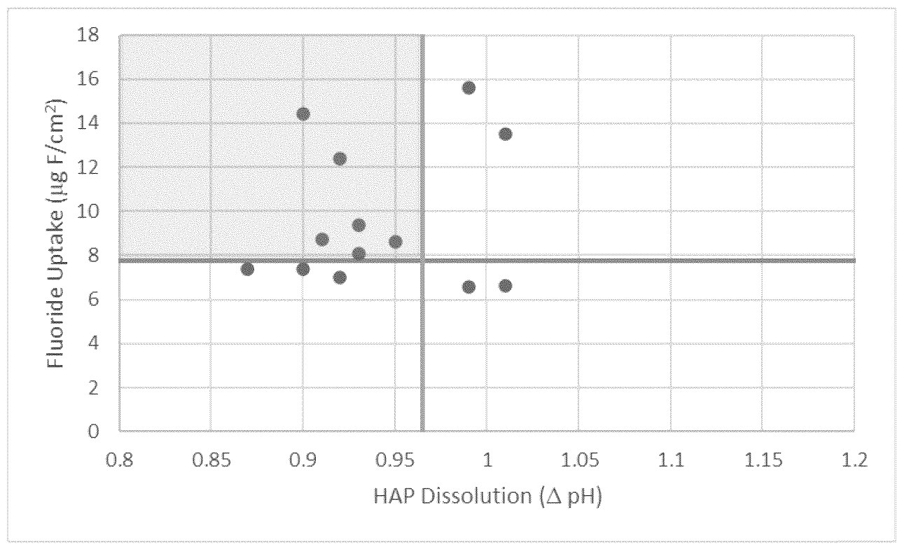

The simultaneous enhancement of fluoride uptake and protection of hydroxyapatite attack is illustrated in FIG. 1. Compositions in the shaded region (i.e., fluoride uptake >7.75 μg F/cm² and HAP dissolution <0.965) were preferred.

In some embodiments, the oral care composition, when exposed with hydroxyapatite to 1% citric acid at pH of about 2.0, has a change in pH (ΔpH) of 0.98 or less, 0.95 or less, or 0.92 or less. In some embodiments, the oral care composition may have a change in L*(ΔL*) of at least 10, preferably at least 20, more preferably at least 25, as measured using a chemical pellicle cleaning ratio (C-PCR) method. In some embodiments, the oral care composition may have a fluoride uptake (μg F/cm²) in a range of from about 6 to about 20, from about 7 to about 20, from about 9 to about 16, or from about 10 to about 16. The oral care composition may have a fluoride uptake (μg F/cm²) in a range of at least about 7, at least about 9, at least about 12 or at least about 14.

The terms “substantially,” “essentially,” “about,” “approximately,” and the like, as may be used herein, represent the inherent degree of uncertainty that may be attributed to any quantitative comparison, value, measurement, or other representation. These terms also represent the degree by which a quantitative representation may vary from a stated reference without resulting in a change in the basic function of the subject matter at issue. Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
a stannous ion source;
a source of ethylenediaminetetraacetate; and
a ligand comprising a dicarboxylate source, a gluconate source, a citrate source, or a combination thereof, preferably wherein the dicarboxylate source comprises an oxalate source or a malonate source, more preferably wherein the dicarboxylate source comprises the oxalate source.

2. The composition of claim 1, wherein a molar ratio of stannous ion to the ligand is about 1:1 to about 1:5.

3. The composition of claim 1, wherein the ligand comprises a monodentate ligand and a polydentate ligand, and a molar ratio of stannous ion to the monodentate ligand to the polydenate ligand is about 1:1:1 to 1:2:5.

4. The composition of claim 1, wherein a pH of the composition is about 4 to less than 6.5.

5. The composition of claim 1, wherein the composition has a change in L*(ΔL*) of at least 10, as measured using a chemical pellicle cleaning ratio (C-PCR) method.

6. The composition of claim 1, wherein the composition has a change in pH (ΔpH) of 0.98 or less when exposed with hydroxyapatite to 1% citric acid at pH of about 2.0.

7. The composition of claim 1, wherein the composition has a fluoride uptake of at least 7 μg F/cm².

8. The composition of claim 1, wherein the composition is essentially free of or, preferably, free of at least one of diethylenetriaminepentaacetic acid etidronic acid.

9. The composition of claim 1, further comprising a fluoride ion source selected from stannous fluoride, sodium fluoride, sodium monofluorophosphate, amine fluoride, and combinations thereof.

10. The composition of claim 1, further comprising at least one of:
a zinc ion source, selected from zinc citrate, zinc lactate, zinc oxide, zinc phosphate and combinations thereof;
postassium nitrate;
a silica abrasive;
a calcium abrasive selected from calcium carbonate, calcium pyrophosphate, calcium phosphate, hydroxyapatite and combinations thereof;
an amino acid selected from glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine and combinations thereof; and
a humectant selected from glycerin, sorbitol, erythritol, xylitol, butylene glycol, propylene glycol, polyethylene glycol and combinations thereof.

11. An oral care composition comprising:
a stannous ion source; and
a source of ethylenediaminetetraacetate (EDTA),
wherein the composition has a pH in a range of from about 6 to about 8, and
wherein the composition has a molar ratio of stannous ions to EDTA in a range of about 1:0.5 to about 1:2.5.

12. The composition of claim 11, further comprising oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, methylmalonic acid, dimethylmalonic acid, tartronic acid, mesoxalic acid, dihydroxymalonic acid, fumaric acid, terephthalic acid, salts thereof, or combinations thereof.

13. The composition of claim 11, wherein the composition has a change in L*(ΔL*) of at least 10, preferably at least 20, more preferably at least 25, as measured using a chemical pellicle cleaning ratio (C-PCR) method.

14. The composition of claim 11, wherein the composition, when exposed with hydroxyapatite to 1% citric acid at pH of about 2.0, has a change in pH (ΔpH) of 0.98 or less, preferably 0.95 or less, more preferably 0.92 or less.

15. The composition of claim 11, wherein the composition has a fluoride uptake of at least 7 μg F/cm², preferably at least about 9 μg F/cm², more preferably at least about 12 μg F/cm².

16. The composition of claim 11, further comprising a fluoride ion source selected from stannous fluoride, sodium fluoride, sodium monofluorophosphate, amine fluoride, and combinations thereof.

17. The composition of claim 11, further comprising a zinc ion source selected from zinc citrate, zinc lactate, zinc oxide, zinc phosphate and combinations thereof.

18. The composition of claim 11, further comprising potassium nitrate.

19. The composition of claim 11, further comprising at least one of a silica abrasive and a calcium abrasive.

20. The composition of claim 19, wherein the silica abrasive comprises precipitated silica and the calcium abrasive comprises calcium carbonate, calcium pyrophosphate, calcium phosphate, hydroxyapatite, or a combination thereof.
